(19) United States
(12) Patent Application Publication
(10) Pub. No.: US 2022/0227934 A1
(43) Pub. Date: Jul. 21, 2022

(54) Compound and Polyamide-Based Polymer Using Same

(71) Applicants: SK Innovation Co., Ltd., Seoul (KR); SK ie technology Co., Ltd., Seoul (KR)

(72) Inventors: Joo Hyun Lee, Daejeon (KR); Hye Ri Kim, Daejeon (KR); Seung Min Jeon, Daejeon (KR); Hyo Shin Kwak, Daejeon (KR)

(21) Appl. No.: 17/575,761

(22) Filed: Jan. 14, 2022

(30) Foreign Application Priority Data
Jan. 15, 2021 (KR) 10-2021-0005986
Nov. 25, 2021 (KR) 10-2021-0164025
Jan. 4, 2022 (KR) 10-2022-0000997

(51) Int. Cl. C08G 69/32; C08G 69/42

(52) U.S. Cl. CPC C08G 69/32; C08G 69/42

(57) ABSTRACT

The present disclosure relates to a compound used to prepare a polyamide-based polymer. More specifically, the present disclosure relates to a compound represented by the following Formula 1 and a polyamide-based polymer using the same.

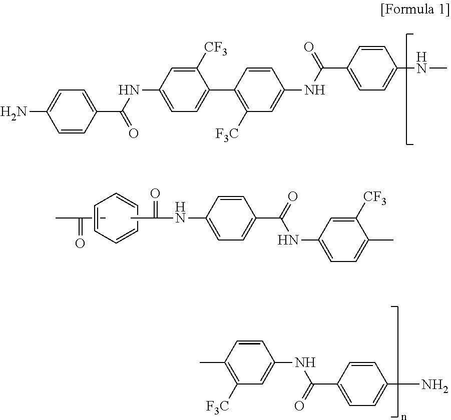

wherein n is an integer of 1 or 2.

### CROSS-REFERENCE TO RELATED APPLICATIONS

This application claims priority to Korean Patent Application Nos. 10-2021-0005986 filed Jan. 15, 2021, 10-2021-0164025 filed Nov. 25, 2021, and 10-2022-0000997 filed Jan. 4, 2022, the disclosures of which are incorporated herein by reference in their entirety.

### BACKGROUND OF THE INVENTION

### Field of the Invention

The following disclosure relates to a compound useful for the preparation of a polyamide-based polymer, and a polyamide-based polymer using the same.

### Description of Related Art

The thin display devices have been implemented in the form of a touch screen panel, and used in various smart devices, including smartphones, tablet personal computers (PCs), and various wearable devices.

Display devices using such a touch screen panel include a window cover including a tempered glass or plastic film on a display panel to protect the display panel from scratches or external impact.

Such a window cover is a component formed at the outermost part of the display device, and thus, heat resistance, mechanical properties, and optical properties should be satisfied, and it is particularly important that the display quality is high and that distortion caused by light, such as a mura phenomenon and image distortion does not occur.

A polyimide-based resin has been used as a polymer material applied to such a window cover film, and in order to be applicable to a foldable display, etc., improvement of mechanical properties while being transparent has been demanded.

### SUMMARY OF THE INVENTION

In one embodiment, provided herein is a compound useful for the preparation of a polyamide-based polymer.

In another embodiment, provided herein is a polyamide including a repeating unit of a novel structure.

In another embodiment, provided herein is a polyamide-based polymer having excellent optical and mechanical properties prepared using the compound, and a polyamide-based film using the same.

In one general aspect, there is provided a compound represented by the following Formula 1.

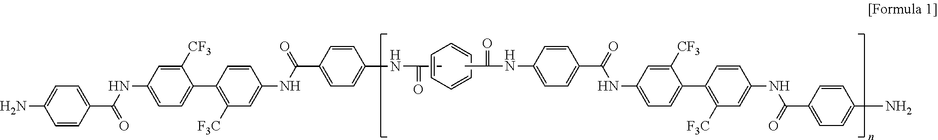

wherein n is an integer of 1 to 10.

The compound represented by Formula 1 may be specifically represented by Formula 2 below:

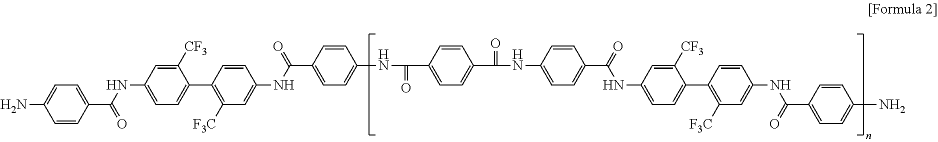

wherein n is an integer of 1 to 10.

In Formulas 1 and 2, n may be an integer of 1 to 5, and more specifically, an integer of 1 or 2.

In another general aspect, there is provided a polyamide having a repeating unit represented by the following Formula 3:

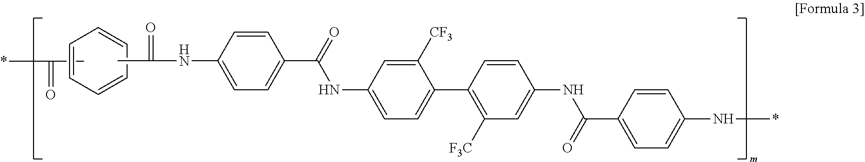

wherein m is an integer selected from 3 to 5,000.

In Formula 3, m may be an integer selected from 10 to 5,000.

The polyamide may be end-capped with units derived from terephthaloyl dichloride or isophthaloyl dichloride at one or more ends.

The polyamide is end-capped with units derived from a compound represented by the following Formula 4 at both ends:

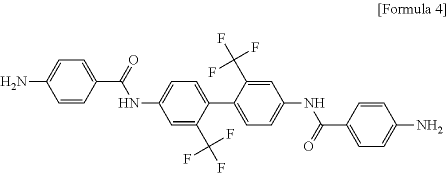 [SMILES: Nc1ccc(C(=O)Nc2ccc(-c3ccc(NC(=O)c4ccc(N)cc4)cc3C(F)(F)F)c(C(F)(F)F)c2)cc1]

In another general aspect, there is provided a polymer obtained using the compound as described above.

In another general aspect, there is provided a polymer obtained using the polyamide as described above.

### DESCRIPTION OF THE INVENTION

Hereinafter, the present disclosure will be described in more detail. The following specific examples and Examples are only a reference for describing the present disclosure in detail, and the present disclosure is not limited thereto, and may be implemented in various forms.

In addition, all technical terms and scientific terms have the same meanings as those commonly understood by a person skilled in the art to which the present disclosure pertains unless otherwise defined. The terms used in the description of the present disclosure are only for effectively describing certain embodiments, and are not intended to limit the present disclosure.

In addition, singular forms used in the detailed description and the claims are intended to include the plural forms unless otherwise indicated in context.

Unless explicitly described to the contrary, “including” any component will be understood to imply the inclusion of other components rather than the exclusion of other components.

Hereinafter, unless specifically defined herein, the term “compound” is a concept including all of a single molecule, an oligomer, and a polymer.

Hereinafter, unless specifically defined herein, the term “compound” is a concept including both an oligomer and a polymer. The oligomer may have a weight average molecular weight in the range of 1000 to 10,000 g/mol, and the polymer may have a weight average molecular weight of 10,000 g/mol or more, and more specifically, 10,000 to 500,000 g/mol.

Unless otherwise defined herein, a polymer includes a homopolymer and a copolymer, and the copolymer includes an alternating polymer, a block copolymer, a random copolymer, a branched copolymer, a crosslinked copolymer, or all of the aforementioned.

“*” as used herein referes to a moiety connected to the same or different atoms or Formulas.

Hereinafter, unless otherwise defined herein, the polyamide-based polymer refers to a resin including a structural unit having an amide bond, and may also include a resin including a structural unit having an amide bond and a structural unit having an imide bond, that is, a polyamideimide resin.

In order to increase the mechanical properties of the polyamide-based film, a polyamide-based polymer including a structural unit derived from a compound having a rigid structure has been conventionally used. Accordingly, however, as compactness between the resins is increased, the retardation (Rₜₕ) in a thickness direction is increased during film formation, and thus, there was a problem in that optical properties deteriorate, for example, distortion occurs, total light transmittance is lowered, and yellowness is greatly increased when applied to displays, etc. Accordingly, there is a need for a polyamide-based polymer capable of imparting excellent mechanical properties while maintaining optical properties.

According to an embodiment, it is possible to impart excellent mechanical properties while maintaining optical properties by using a diamine compound including an amide bond (—CONH—) in a molecule obtained by reacting an aromatic diamine with an aromatic diacid dichloride, and a polyamide-based polymer using the same.

Specifically, the compound may be represented by the following Formula 1:

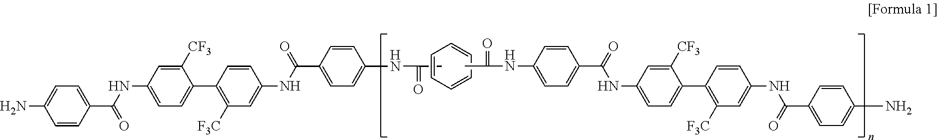

wherein n may be an integer of 1 to 10, but it is not necessarily limited thereto.

Although not wishing to be bound by a particular theory, since the compound represented by Formula 1 includes a plurality of amide bonds in the molecule and also includes amine groups at both ends, when the compound represented by Formula 1 is reacted with a dianhydride to prepare a polyamide-based polymer, the intramolecular interaction and/or intermolecular interaction of the amide bond may be increased to significantly improve mechanical properties.

In addition, although not wishing to be bound by a particular theory, since the compound represented by Formula 1 includes an aromatic ring, for example, a benzene ring, the carbon content of the resin may be increased, and the resin may have a more rigid structure. Accordingly, it is possible to provide a polyamide-based polymer having sufficient optical properties while having superior mechanical properties, and a polyamide-based film using the same.

For example, the compound represented by Formula 1 is obtained by reacting a compound represented by the following Formula 4 (hereinafter, referred to as AB-TFMB) with terephthaloyl dichloride (TPC) or isophthaloyl dichloride (IPC), but it is not necessarily limited thereto:

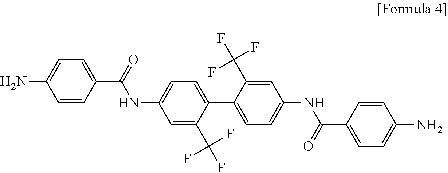 [SMILES: Nc1ccc(C(=O)Nc2ccc(-c3ccc(NC(=O)c4ccc(N)cc4)cc3C(F)(F)F)c(C(F)(F)F)c2)cc1]

For example, the compound represented by Formula 1 may be obtained by reacting the AB-TFMB with TPC or IPC in a molar ratio of 1 to 3:1, more specifically 1.5 to 2:1, but it is not necessarily limited thereto.

More specifically, the compound may be a compound represented by the following Formula 2:

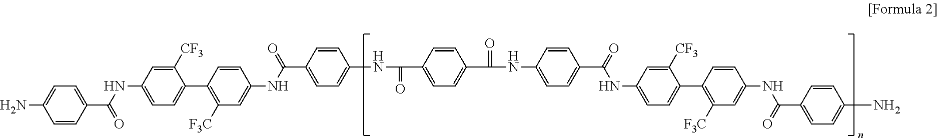

wherein n may be an integer of 1 to 10, but it is not necessarily limited thereto.

For example, n may be an integer of 1 to 5, for example, may be an integer of 1 to 3, for example, may be 1 or 2, but it is not necessarily limited thereto.

For example, the compound represented by Formula 2 is obtained by reacting a compound represented by Formula 4 (hereinafter, referred to as AB-TFMB) with terephthaloyl dichloride (TPC), but it is not necessarily limited thereto.

For example, the compound represented by Formula 1 or Formula 2 may be applied as a diamine monomer for preparing a polyamide-based polymer. The polyamide-based polymer prepared including the compound represented by Formula 1 or Formula 2 and a film using the same may impart improved mechanical properties while maintaining optical properties as compared to a film not containing the compound.

Hereinafter, the compound represented by Formula 1 or Formula 2 may be a first aromatic diamine monomer, and for example, the polyamide-based polymer may further include a different second aromatic diamine monomer in addition to a compound represented by Formula 1 or Formula 2. For example, the second aromatic diamine monomer may be a known diamine monomer commonly used in the art, and as long as the effect of the present disclosure is obtained, the type thereof is not limited, but a fluorine substituent may be introduced, and the polyamide-based polymer and a film using the same may provide more excellent optical properties by using the aromatic diamine monomer into which a fluorine substituent is introduced.

Specifically, the second aromatic diamine monomer may include an aromatic ring substituted with one or two or more trifluoroalkyl groups. For example, the aromatic ring substituted with the trifluoroalkyl group may be further unsubstituted or substituted with a substituent other than the trifluoroalkyl group, but it is not necessarily limited thereto.

More specifically, the second aromatic diamine monomer may include 2,2′-bis(trifluoromethyl)-benzidine (hereinafter, also referred to as TFMB), but it is not necessarily limited thereto.

For example, when the polyamide-based polymer is prepared, the content of the compound represented by Formula 1 or Formula 2 may be 0.1 to 99.9 mol %, more specifically 0.5 to 99.5 mol %, further more specifically 1 to 99 mol %, and further still more specifically 2 to 98 mol % of the total content of the aromatic diamine, but it is not necessarily limited thereto.

The polyamide-based polymer may be prepared by reacting an aromatic diamine including the compound represented by Formula 1 or Formula 2 alone or an aromatic diamine including a mixture of a compound represented by Formula 1 or Formula 2 and a different type of known second aromatic diamine monomer, with a known dianhydride.

The dianhydride may include any one or two or more selected from an aromatic dianhydride and a cycloaliphatic dianhydride, but it is not necessarily limited thereto.

The aromatic dianhydride means a dianhydride including at least one aromatic ring, wherein the aromatic ring may be a single ring; may be a fused ring in which two or more aromatic rings are fused; or may be a non-fused ring in which two or more aromatic rings are connected by a single bond, a substituted or unsubstituted C₁-C₅ alkylene group, O, or C(═O), but is not necessarily limited thereto.

Specifically, the aromatic dianhydride may include a benzene ring, a non-fused ring in which two or more benzene rings are connected by a single bond, a non-fused ring in which two or more benzene rings are connected by a methylene group substituted with one or more trifluoromethyl groups, or a dianhydride including a combination thereof, but it is not necessarily limited thereto.

More specifically, the aromatic dianhydride may include 2,2′-bis-(3,4-dicarboxylphenyl) hexafluoropropane dianhydride (6FDA), 3,3′,4,4′-biphenyltetracarboxylic dianhydride (BPDA), 9,9-bis(3,4-dicarboxyphenyl)fluorene dianhydride (BPAF), oxydiphthalic dianhydride (ODPA), sulfonyl diphthalic anhydride (SO2DPA), (isopropylidenediphenoxy)bis (phthalic anhydride) (6HDBA), 4-(2,5-dioxotetrahydrofuran-3-yl)-1,2,3,4-tetrahydronaphthalene-1,2-dicarboxylic dianhydride (TDA), 1,2,4,5-benzene tetracarboxylic dianhydride (PMDA), benzophenone tetracarboxylic dianhydride (BTDA), bis(carboxyphenyl)dimethyl silane dianhydride (SiDA), bis(dicarboxyphenoxy)diphenyl sulfide dianhydride (BDSDA), or a combination thereof, but it is not necessarily limited thereto.

More specifically, the aromatic dianhydride may include 2,2′-bis-(3,4-dicarboxylphenyl) hexafluoropropane dianhydride (6FDA), 3,3′,4,4′-biphenyltetracarboxylic dianhydride (BPDA), or a combination thereof. For example, the aromatic dianhydride may include, but is not limited to, 2,2′-bis-(3,4-dicarboxylphenyl) hexafluoropropane dianhydride (6FDA), and may further include the above-described aromatic dianhydride if necessary, but it is not necessarily limited thereto.

The cycloaliphatic dianhydride means a dianhydride including at least one substituted or unsubstituted C₃-C₆₀ aliphatic ring, and the substituted or unsubstituted C₃-C₆₀ aliphatic ring may include a substituted or unsubstituted C₃-C₆₀ cycloalkane, a substituted or unsubstituted C₃-C₆₀ cycloalkene, or a combination thereof, but it is not necessarily limited thereto.

Specifically, the cycloaliphatic dianhydride may include a dianhydride including substituted or unsubstituted cyclobutane, substituted or unsubstituted cyclopentane, substituted or unsubstituted cyclohexane, substituted or unsubstituted cycloheptane, substituted or unsubstituted cyclooctane, substituted or unsubstituted cyclobutene, substituted or unsubstituted cyclopentene, substituted or unsubstituted cyclohexene, substituted or unsubstituted cycloheptene, substituted or unsubstituted cyclooctene, or combinations thereof, but it is not necessarily limited thereto.

More specifically, the cycloaliphatic dianhydride may include 1,2,3,4-cyclobutanetetracarboxylic dianhydride (CBDA), 5-(2,5-dioxotetrahydrofuryl)-3-methylcyclohexene-1,2-dicarboxylic dianhydride (DOCDA), bicyclo[2.2.2]oct-7-ene-2,3,5,6-tetracarboxylic dianhydride (BTA), bicyclooctene-2,3,5,6-tetracarboxylic dianhydride (BODA), 1,2,3,4-cyclopentanetetracarboxylic dianhydride (CPDA), 1,2,4,5-cyclohexanetetracarboxylic dianhydride (CHDA), 1,2,4-tricarboxy -3-methylcarboxycyclopentane dianhydride (TMDA), 1,2,3,4-tetracarboxycyclopentane dianhydride (TCDA), or a combination thereof, but it is not necessarily limited thereto.

For example, the cycloaliphatic dianhydride may include 1,2,3,4-cyclobutanetetracarboxylic dianhydride (CBDA), but it is not necessarily limited thereto, and may further include the above-described cycloaliphatic dianhydride, if necessary.

For example, when the polyamide-based polymer is prepared, the aromatic diamine and the dianhydride may be used in an equivalent ratio of 1:0.9 to 1.1, and more specifically, approximately to 1:1, but it is not necessarily limited thereto.

According to another embodiment, a polyamide having a repeating unit represented by the following Formula 3 is provided. In this case, the polyamide includes an oligomer or a polymer.

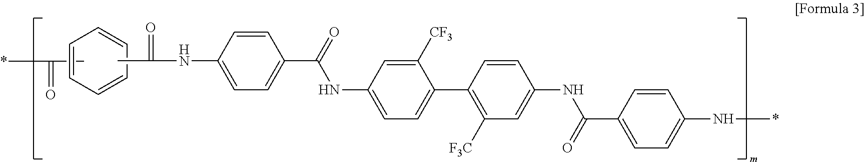

wherein m may be an integer selected from 3 to 5,000, but it is not necessarily limited thereto.

In Formula 3, the polyamide may be end-capped with units derived from terephthaloyl dichloride or isophthaloyl dichloride at one or more ends. Alternatively, the polyamide may be end-capped with units derived from a compound (AB-TFMB) represented by the following Formula 4 at both ends.

For example, m may be an integer selected from 5 to 5,000, for example, from 10 to 5,000, but it is not necessarily limited thereto.

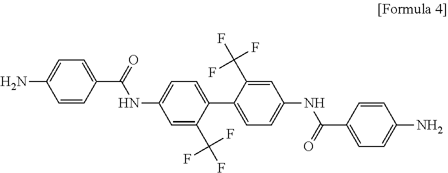 [SMILES: Nc1ccc(C(=O)Nc2ccc(-c3ccc(NC(=O)c4ccc(N)cc4)cc3C(F)(F)F)c(C(F)(F)F)c2)cc1]

The polyamide represented by Formula 3 may be obtained by reacting the compound represented by Formula 4 (hereinafter, referred to as AB-TFMB) with terephthaloyl dichloride (TPC) or isophthaloyl dichloride (IPC). For example, the compound represented by Formula 3 may be obtained by reacting the AB-TFMB with TPC or IPC in a molar ratio of 1 to 10:1, more specifically 1.01 to 8:1, but it is not necessarily limited thereto.

For example, to explain the method for preparing the compounds represented by Formulas 1 to 3, the above-described AB-TFMB and TPC or IPC may be reacted in the presence of an organic solvent. The organic solvent may be used without limitation as long as it can dissolve the monomers. Specifically, for example, the organic solvent may be any one or more polar solvents selected from the group consisting of dimethylacetamide (DMAc), N-methyl-2-pyrrolidone (NMP), dimethylformamide (DMF), dimethylsulfoxide (DMSO), ethyl cellosolve, methyl cellosolve, acetone, ethyl acetate, m-cresol, gamma-butyrolactone (GBL), and derivatives thereof, but it is not limited thereto.

In addition, the reaction may be carried out using a reaction catalyst such as pyridine.

In this case, depending on the molar ratio of the reaction between AB-TFMB and TPC or IPC, compounds selected from Formulas 1 to 3 may be obtained.

The reaction may be carried out at room temperature, specifically, 20 to 30° C., and the reaction may proceed in an inert atmosphere such as argon or nitrogen during the reaction, but it is not necessarily limited thereto.

According to another embodiment, there is provided a composition containing the above-described polyamide-based polymer and a polyamide-based polymer including a solvent.

The composition containing the polyamide-based polymer may further include an additive, if necessary, in addition to the above-described polyamide-based polymer. The additive may be for improving film formation, adhesion, optical properties, mechanical properties, flame resistance, etc., and may be, for example, a flame retardant, an adhesion enhancer, an inorganic particle, an antioxidant, a UV inhibitor, and/or a plasticizer, but it is not necessarily limited thereto.

According to another embodiment, there is provided a polyamide-based film including the above-described polyamide-based polymer.

For example, the polyamide-based film may simultaneously satisfy physical properties of a modulus of 6 GPa or more, a total light transmittance at 400 to 700 nm as measured according to ASTM D1003 of 88% or more, a haze of 1.5% or less as measured according to ASTM D1003, and yellowness index of 6 or less as measured according to ASTM E313, but it is not necessarily limited thereto.

More specifically, the polyamide-based film may simultaneously satisfy physical properties of a modulus of 6 GPa or more, specifically GPa or more, and more specifically 6 to 10 GPa, a total light transmittance at 400 to 700 nm as measured according to ASTM D1003 of 88% or more, and specifically, 89% or more, a haze of 1.5% or less, specifically, 1.0% or less, and more specifically, 0.5% or more as measured according to ASTM D1003, and yellowness index of 6 or less, specifically, 5 or less, and more specifically, 4 or more as measured according to ASTM E313, but it is not limited thereto.

For example, the polyamide-based film may have a thickness of 1 to 500 μm, for example, 10 to 250 μm, for example, 10 to 100 μm, but it is not necessarily limited thereto.

Hereinafter, the present disclosure will be described in more detail on the basis of Examples and Comparative Examples. However, the following Examples and Comparative Examples are only examples for describing the present disclosure in more detail, and it is not limited by the following Examples and Comparative Examples.

[Measurement Method of Physical Properties]

(1) Weight Average Molecular Weight

The weight average molecular weight was measured by dissolving the film in DMAc eluent containing 0.05 M LiCl. Measurement was carried out using gel permeation chromatography (GPC) (Waters GPC system, Waters 1515 isocratic HPLC Pump, Waters 2414 Refractive Index detector), a column was connected to Olexis, Polypore, and mixed D columns, polymethylmethacrylate (PMMA STD) was used as the standard, and the analysis was carried out at 35° C. at flow rate of 1 mL/min.

(2) Modulus

A modulus was measured according to ASTM D882 using UTM 3365 (Instron Corp.) under conditions of pulling a polyamide-imide film having a length of 50 mm and a width of 10 mm at 50 mm/min at 25° C.

Preparation of Compound

### Synthesis Example 1

Under a nitrogen environment, 250 ml of N,N-dimethylacetamide and 14.2 g (2 eq.) of pyridine were added to 50 g of AB-TFMB and dissolved. A solution of 9.1 g (0.5 eq.) of terephthaloyl chloride (TPC) in 100 ml of N,N-dimethylacetamide was added dropwise thereto for 30 minutes.

After stirring at room temperature (25° C.) for 3 hours, the prepared reactant was added dropwise to 3.5 L of distilled water to allow the organics to precipitate. After filtering the solid, the mixture was filtered again with 1 L of distilled water and dried under nitrogen condition to obtain 53 g of compound 1 (yield 95%).

¹H NMR (DMSO-d6, 500 MHz, ppm): 10.72 (brs, 2H), 10.58 (brs, 2H), 10.15 (brs, 2H), 8.38-8.31 (m, 4H), 8.17-8.00 (m, 16H), 7.78- 7.75 (m, 4H), 7.39-7.29 (m, 4H), 7.65-6.62 (m, 4H), 5.83 (s, 4H).

Here, AB-TFMB:TPC was used in a molar ratio of 2:1, and the reaction scheme is as follows:

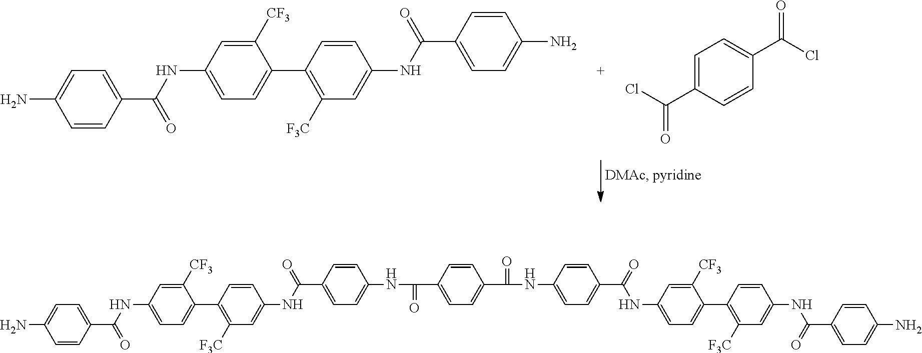 [SMILES: Nc1ccc(C(=O)Nc2ccc(-c3ccc(NC(=O)c4ccc(N)cc4)cc3C(F)(F)F)c(C(F)(F)F)c2)cc1 ; O=C(Cl)c1ccc(C(=O)Cl)cc1 ; Nc1ccc(C(=O)Nc2ccc(-c3ccc(NC(=O)c4ccc(NC(=O)c5ccc(C(=O)Nc6ccc(C(=O)Nc7ccc(-c8ccc(NC(=O)c9ccc(N)cc9)cc8C(F)(F)F)c(C(F)(F)F)c7)cc6)cc5)cc4)cc3C(F)(F)F)c(C(F)(F)F)c2)cc1]

### Synthesis Example 2

Under a nitrogen environment, 250 ml of N,N-dimethylacetamide and 14.2 g (2 eq.) of pyridine were added to 50 g of AB-TFMB and dissolved. A solution of 12.1 g (0.67 eq.) of terephthaloyl chloride (TPC) in 100 ml of N,N-dimethylacetamide was added dropwise thereto for 30 minutes.

After stirring at room temperature (25° C.) for 3 hours, the prepared reactant was added dropwise to 3.5 L of distilled water to allow the organics to precipitate. After filtering the solid, the mixture was filtered again with 1 L of distilled water and dried under nitrogen condition to obtain 55 g of compound 2 (yield 95%).

¹H NMR (DMSO-d6, 500 MHz, ppm): 10.72 (brs, 4H), 10.58 (brs, 4H), 10.15 (brs, 2H), 8.38-8.31 (m, 6H), 8.17-8.00 (m, 30H), 7.78-7.75 (m, 4 H), 7.39-7.29 (m, 6H), 7.65-6.62 (m, 4H), 5.83 (s, 4H).

Here, AB-TFMB:TPC was used in a molar ratio of 3:2, and the reaction scheme is as follows:

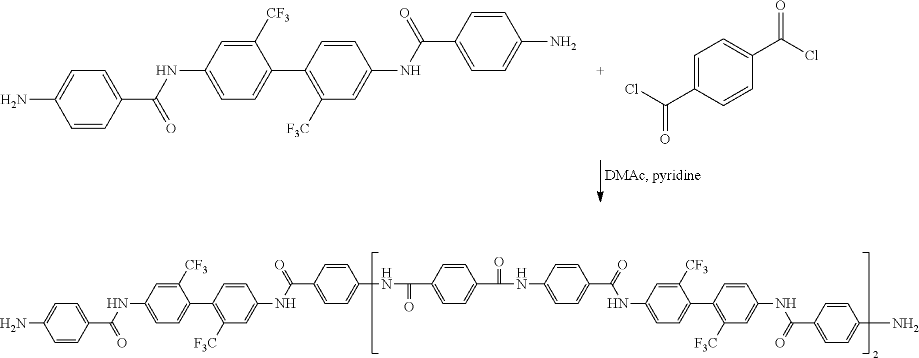

Preparation of Polyamide-Based Polymer

### Example 1

N,N-dimethylacetamide (DMAc, 290 g) and 2.2′-bis(trifluoromethyl)-benzidine (TFMB, 26 g) were added to a reactor under nitrogen atmosphere and stirred sufficiently, and then terephthaloyl dichloride (TPC, 11.8 g) was added thereto and stirred for 6 hours to be dissolved and reacted.

Thereafter, a reaction product obtained by precipitation and filtration using an excess of water was dried under vacuum at 90° C. for 6 hours or more to obtain an oligomer.

Again, N,N-dimethylacetamide (DMAc, 219.35 g), compound 1 (2.486 g) obtained in Synthesis Example 1, the oligomer (15.634 g), AB-TFMB (0.014 g), and 2,2′-bis(trifluoromethyl)-benzidine (TFMB, 0.785 g) were added to a reactor under a nitrogen atmosphere. 2,2′-bis-(3,4-dicarboxylphenyl) hexafluoropropane dianhydride (6FDA, 2.896 g) and 3,3′,4,4′-biphenyltetracarboxylic dianhydride (BPDA, 2.557 g) were sequentially added thereto and dissolved and reacted with stirring at 40° C. for 12 hours to prepare a polyamide-based resin precursor composition. Here, the solid content was adjusted to be 10% by weight, and the temperature of the reactor was maintained at 40° C.

Then, each of pyridine and acetic anhydride was sequentially added to the solution at 2.5 times the moles of the total dianhydride content, and stirred at 60° C. for 12 hours to prepare a composition containing a polyamide-based polymer. The polyamide-based polymer had a weight average molecular weight of 270,000 g/mol.

### Example 2

N,N-dimethylacetamide (DMAc, 290 g) and 2.2′-bis(trifluoromethyl)-benzidine (TFMB, 26 g) were added to a reactor under nitrogen atmosphere and stirred sufficiently, and then terephthaloyl dichloride (TPC, 11.8 g) was added thereto and stirred for 6 hours to be dissolved and reacted.

Thereafter, a reaction product obtained by precipitation and filtration using an excess of water was dried under vacuum at 90° C. for 6 hours or more to obtain an oligomer.

Again, N,N-dimethylacetamide (DMAc, 226.74 g), compound 1 (10.043 g) obtained in Synthesis Example 1, the oligomer (3.553 g), AB-TFMB (0.058 g), and 2,2′-bis(trifluoromethyl)-benzidine (TFMB, 4.978 g) were added to a reactor under a nitrogen atmosphere. 1,2,3,4-cyclobutanetetracarboxylic dianhydride (CBDA, 3.906 g) and 2,2′-bis-(3,4-dicarboxylphenyl) hexafluoropropane dianhydride (6FDA, 2.654 g) were sequentially added thereto and dissolved and reacted with stirring at 40° C. for 12 hours to prepare a polyamide-based resin precursor composition. Here, the solid content was adjusted to be 10% by weight, and the temperature of the reactor was maintained at 40° C.

Then, each of pyridine and acetic anhydride was sequentially added to the solution at 2.5 times the moles of the total dianhydride content, and stirred at 60° C. for 12 hours to prepare a composition containing a polyamide-based polymer. The polyamide-based polymer had a weight average molecular weight of 330,000 g/mol.

### Example 3

N,N-dimethylacetamide (DMAc, 290 g) and 2.2′-bis(trifluoromethyl)-benzidine (TFMB, 26 g) were added to a reactor under nitrogen atmosphere and stirred sufficiently, and then terephthaloyl dichloride (TPC, 11.8 g) was added thereto and stirred for 6 hours to be dissolved and reacted.

Thereafter, a reaction product obtained by precipitation and filtration using an excess of water was dried under vacuum at 90° C. for 6 hours or more to obtain an oligomer.

Again, N,N-dimethylacetamide (DMAc, 229.70 g), compound 2 (6.779 g) obtained in Synthesis Example 2, the oligomer (2.558 g), AB-TFMB (3.064 g), and 2,2′-bis(trifluoromethyl)-benzidine (TFMB, 4.969 g) were added to a reactor under a nitrogen atmosphere. 1,2,3,4-cyclobutanetetracarboxylic dianhydride (CBDA, 3.375 g) and 2,2′-bis-(3,4-dicarboxylphenyl) hexafluoropropane dianhydride (6FDA, 4.778 g) were sequentially added thereto and dissolved and reacted with stirring at 40° C. for 12 hours to prepare a polyamide-based resin precursor composition. Here, the solid content was adjusted to be 10% by weight, and the temperature of the reactor was maintained at 40° C.

Then, each of pyridine and acetic anhydride was sequentially added to the solution at 2.5 times the moles of the total dianhydride content, and stirred at 60° C. for 12 hours to prepare a composition containing a polyamide-based polymer. The polyamide-based polymer had a weight average molecular weight of 320,000 g/mol.

### Example 4

250 ml of N,N-dimethylacetamide and AB-TFMB were added to a reactor under a nitrogen environment and sufficiently stirred. Thereafter, terephthaloyl dichloride (TPC) was added thereto, and the mixture was dissolved and reacted by stirring for 6 hours to prepare a polyamide-based resin composition.

Here, the amount of each monomer was 10 moles of AB-TFMB with respect to 1 mole of TPC, and the solid content was adjusted to be 4% by weight, and the temperature of the reactor was maintained at 30° C.

After completion of the reaction, it was confirmed that a polymer resin including an amide group was prepared.

### Comparative Example 1

N,N-dimethylacetamide (DMAc, 224.56 g) and 2,2′-bis(trifluoromethyl)-benzidine (TFMB, 11.208 g,) were added to a reactor under a nitrogen atmosphere. 1,2,3,4-cyclobutanetetracarboxylic dianhydride (CBDA, 1.366 g) and 2,2′-bis-(3,4-dicarboxylphenyl) hexafluoropropane dianhydride (6FDA, 12.377 g) were sequentially added thereto and dissolved and reacted with stirring at 40° C. for 12 hours to prepare a polyamide-based resin precursor composition. Here, the solid content was adjusted to be 10% by weight, and the temperature of the reactor was maintained at 40° C.

Then, each of pyridine and acetic anhydride was sequentially added to the solution at 2.5 times the moles of the total dianhydride content, and stirred at 60° C. for 12 hours to prepare a composition containing a polyamide-based polymer. The polyamide-based polymer had a weight average molecular weight of 290,000 g/mol.

### Comparative Example 2

N,N-dimethylacetamide (DMAc, 236.85 g), AB-TFMB (19.547 g), and 2,2′-bis(trifluoromethyl)-benzidine (TFMB, 11.208 g) were added to a reactor under a nitrogen atmosphere. 1,2,3,4-cyclobutanetetracarboxylic dianhydride (CBDA, 2.732 g) and 2,2′-bis-(3,4-dicarboxylphenyl) hexafluoropropane dianhydride (6FDA, 12.377 g) were sequentially added thereto and dissolved and reacted with stirring at 40° C. for 12 hours to prepare a polyamide-based resin precursor composition. Here, the solid content was adjusted to be 10% by weight, and the temperature of the reactor was maintained at 40° C.

Then, each of pyridine and acetic anhydride was sequentially added to the solution at 2.5 times the moles of the total dianhydride content, and stirred at 60° C. for 12 hours to prepare a composition containing a polyamide-based polymer. The polyamide-based polymer had a weight average molecular weight of 300,000 g/mol.

### Comparative Example 3

N,N-dimethylacetamide (DMAc, 290 g) and 2.2′-bis(trifluoromethyl)-benzidine (TFMB, 26 g) were added to a reactor under nitrogen atmosphere and stirred sufficiently, and then terephthaloyl dichloride (TPC, 11.8 g) was added thereto and stirred for 6 hours to be dissolved and reacted.

Thereafter, a reaction product obtained by precipitation and filtration using an excess of water was dried under vacuum at 90° C. for 6 hours or more to obtain an oligomer.

Again, N,N-dimethylacetamide (DMAc, 232.19 g), the oligomer (14.923 g) and 2,2′-bis(trifluoromethyl)-benzidine (TFMB, 3.465 g) were added to a reactor under a nitrogen atmosphere. 2,2′-bis-(3,4-dicarboxylphenyl) hexafluoropropane dianhydride (6FDA, 4.054 g) and 3,3′,4,4′-biphenyltetracarboxylic dianhydride (BPDA, 3.356 g) were sequentially added thereto and dissolved and reacted with stirring at 40° C. for 12 hours to prepare a polyamide-based resin precursor composition. Here, the solid content was adjusted to be 10% by weight, and the temperature of the reactor was maintained at 40° C.

Then, each of pyridine and acetic anhydride was sequentially added to the solution at 2.5 times the moles of the total dianhydride content, and stirred at 60° C. for 12 hours to prepare a composition containing a polyamide-based polymer. The polyamide-based polymer had a weight average molecular weight of 320,000 g/mol.

Manufacture of Film

The polyamide-based resin compositions of Examples 1 to 3 and Comparative Examples 1 to 3 were each subjected to solution-casting on a glass substrate using an applicator. Thereafter, after primary drying for 30 minutes at 90° C. using a convection oven, additional heat treatment was carried out at 280° C. for 1 hour under nitrogen stream conditions, followed by cooling at room temperature. Then, a film formed on the glass substrate was separated from the substrate to obtain a polyamide film.

The modulus of the manufactured film was measured and shown in Table 1 below.

Evaluation: Modulus

TABLE 1

| Division | Thickness (μm) | Modulus (GPa) |
|---|---|---|
| Example 1 | 48 | 6.47 |
| Example 2 | 47.8 | 9.08 |
| Example 3 | 50 | 7.75 |
| Comparative | 48 | 4.03 |
| Example 1 | | |
| Comparative | 49 | 5.9 |
| Example 2 | | |
| Comparative | 51 | 5.8 |
| Example 3 | | |

It can be confirmed from Table 1 that a polyamide-based film manufactured from the polyamide-based polymer of Examples 1 to 3 had a higher modulus than the polyamide-based film manufactured from the polyamide-based polymer of Comparative Example, and thus had excellent mechanical properties.

Hereinabove, although the present disclosure has been described by specific matters and the limited embodiments, they have been provided only for assisting in a more general understanding of the present disclosure. Therefore, the present disclosure is not limited to the exemplary embodiments. Various modifications and changes may be made by those skilled in the art to which the present disclosure pertains from this description.

Therefore, the spirit of the present disclosure should not be limited to the above-mentioned embodiments, but the claims and all of the modifications equal or equivalent to the claims are intended to fall within the scope and spirit of the present disclosure.

1. 1A compound represented by the following Formula 1: 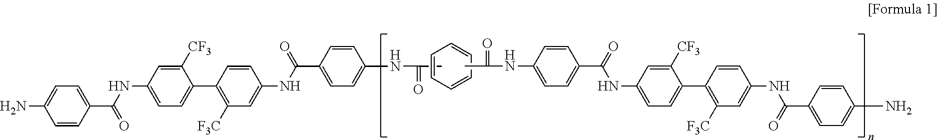
wherein n is an integer of 1 to 10.

2. The compound of claim 1,
wherein the compound is represented by the following Formula 2: 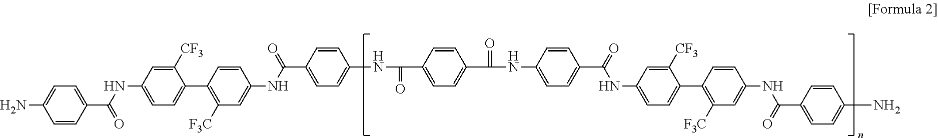
wherein n is an integer of 1 to 10.

3. The compound of claim 1,
wherein n is an integer of 1 to 5.

4. The compound of claim 1,
wherein n is an integer of 1 or 2.

5. A polyamide comprising a repeating unit represented by the following Formula 3: 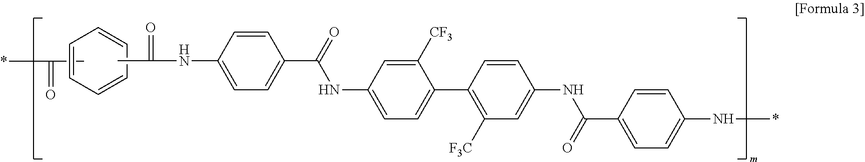
wherein m is an integer selected from 3 to 5,000.

6. The polyamide of claim 5,
wherein m is an integer selected from 10 to 5,000.

7. The polyamide of claim 5,
wherein the polyamide is end-capped with units derived from terephthaloyl dichloride or isophthaloyl dichloride at one or more ends.

8. The polyamide of claim 5,
wherein the polyamide is end-capped with units derived from a compound represented by the following Formula 4 at both ends:

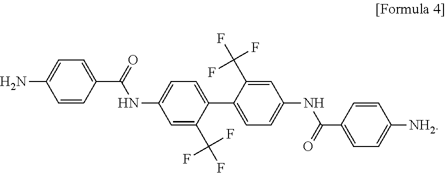 [SMILES: Nc1ccc(C(=O)Nc2ccc(-c3ccc(NC(=O)c4ccc(N)cc4)cc3C(F)(F)F)c(C(F)(F)F)c2)cc1]

9. A polymer obtained using the compound of claim 1.

10. The polymer obtained using the polyamide of claim 5.
